(19) United States
(12) Patent Application Publication
(10) Pub. No.: US 2008/0260864 A1
(43) Pub. Date: Oct. 23, 2008

(54) Topical Compositions for the Treatment of Depilation-Induced Irritation or Prevention Thereof

(75) Inventor: Avi Dascalu, Tel-Aviv (IL)

Correspondence Address: MOORE & VAN ALLEN PLLC, P.O. BOX 13706, Research Triangle Park, NC, 27709, US

(73) Assignee: MEDIDERMIS LTD., Netanya (IL)

(21) Appl. No.: 12/067,219

(22) PCT Filed: Oct. 18, 2006
(86) PCT No.: PCT/IL2006/001198

§ 371 Date: Jun. 25, 2008

Related U.S. Application Data
(60) Provisional application No. 60/728,562, filed on Oct. 21, 2005.

(51) Int. Cl. A61K 33/06; A61P 17/00

(52) U.S. Cl. 424/685

(57) ABSTRACT

The present invention relates to topical compositions comprising as active ingredients Aluminium Chloride and Sulfur for treatment of skin irritation induced by depilation. Said compositions have pharmaceutical and/or cosmetic and/or quasi cosmetic properties. The present invention also involves methods of making and methods of using such compositions. Furthermore, the present invention consists of a method for prevention and/or treatment of skin irritation induced by depilation.

### FIELD OF THE INVENTION

The present invention relates to topical compositions for treatment of skin irritation induced by depilation. Said compositions have pharmaceutical and/or cosmetic and/or quasi cosmetic properties. The present invention also involves methods of making and methods of using such compositions. Furthermore, the present invention consists of a method for prevention and/or treatment of skin irritation induced by depilation.

### BACKGROUND OF THE INVENTION

Depilation of body areas is a widespread cosmetic procedure. Unwanted hair is treated by women mostly, and in the last years by men as well (Facial and Body Hair, R Dowber. In Cosmetic Dermatology, Eds R Baran and HI Maibach, Martin Dunitz Ltd, 1994, pp 139). The depilation procedure is performed by different methods, e.g.
1. Mechanical: shaving or hair plucking,
2. Chemical: such as waxes, sugaring or depilatory products,
3. Electrical: electrolysis, or
4. Light energy, such as laser or intense pulse lightening.

The areas of the body involved in the depilation procedure are various, mostly the armpits and legs, to be followed by facial areas, groins, arms and chest.

Actually, the skin is covered with millions (×5) of hair follicles (Hair Diseases. In Habif: Clinical Dermatology, 4th ed., Copyright © 2004 Mosby, Inc. pp 834). Depilation of a skin area by any of the before mentioned methods might causes a peculiar irritation of the skin due to direct damage to part of the thousands of hair follicles involved in the procedure. This might be due to a minimal injury inflicted upon the outer epidermis, from stratum corneum to stratum granulosum, either through repeated mechanical plucking of hair follicles, skin contact with various chemicals or heat damage, depending on the depilation technique.

Clinically, the depilation-induced skin irritation is perceived as skin redness and itching or dry skin due to repetitive damage. These symptoms are generally treated by either
1. Non specific general purpose emollient creams and lotions. These remedies are based on various chemicals, such as urea, lactic acid and aloe vera extracts. These low potency remedies are used sporadically with limited effects.
2. Low or medium potency steroids might be applied with satisfactory effects. However, single or recurrent treatment of skin areas with steroidal products might cause skin damage and/or recurrent infections caused by a shift in the microbial population, e.g. overpopulation by Staphylococcus Aureus, and/or skin atrophy.

Therefore, depilation causes undue personal damage in a large number of cases and an effective and tailored, yet simple, remedy to skin irritation is not available, yet. There is a need to provide a remedy for depilation-induced skin irritation which:
1. Can be used topical;
2. Can be applied easily and is completely absorbed by the skin;
3. Reduces dramatically the skin irritation due to depilation; and
4. Is safe and devoid of side effects.

It is an object of the subject invention to provide topical compositions which treat skin irritation induced by various depilatory procedures.

It is an object of the subject invention to provide topical compositions, which prevent skin irritation induced by various depilatory procedures.

It is an object of the subject invention to provide methods for prevention and treatment of skin irritation induced by various depilatory procedures.

### PRIOR ART

I. Aluminium Chloride, or aluminium chlorohydrate, CASRN: 7446-70-0, EINECS no 215-477-2. It is a complex inorganic salt, loosely hydrated and encompasses a range of aluminium to chloride ratios. Aluminium chloride exists in two categories: either as anhydrous, suitable as an acid catalyst or as a hexahydrate form (The Merck Index, 12^{th} Ed., Merck & Co., Inc., 1996, pp 60).

It functions as a
1. Cosmetic: (International Cosmetic Ingredient Dictionary and Handbook, Wenninger et al., The Cosmetic, Toiletry and Fragrance Association, 8th Edition, 2000, pp 54)
   a. Antiperspirant Agent,
   b. Cosmetic Astringent and
   c. Deodorant Agent
2. Medicated topical aluminium chloride, commonly employed for treatment of hyperhidrosis at 20-25% concentrations (Andrew's Diseases of the Skin, Eds. R B Odom, W D James, T. G. Berger, 9^{th} Ed. , W. B. Saunders, 2000, pp 973). The mechanism of action is related to obstruction of sweat pores and induction of atrophy of secretory cells within the sweat glands (Palmoplantar hyperhidrosis: a therapeutic challenge. Thomas I, Brown J, Vafaie J, Schwartz R A. Am Fam Physician 2004; 69:1117-20).
   II. Sulfur, an inorganic naturally occurring element. CAS no 7704-34-9, EINECS no 231-722-6. It functions as an Anti Acne, Anti Dandruff, Hair Conditioning and Skin Conditioning agent. Sulfur is used in various skin products, and its basic mechanism of work is unknown (International Cosmetic Ingredient Dictionary and Handbook, Wenninger et al., The Cosmetic, Toiletry and Fragrance Association, 8th Edition, 2000, pp. 1456).

### DISCLOSURE OF THE INVENTION

It has now surprisingly been found that application of a topical combination of Aluminium Chloride (AlCl) together with Sulfur treats and prevents the irritation induced by depilation.

### DETAILED DESCRIPTION OF THE INVENTION

A search of the literature did not reveal any reference to AlCl as an anti irritant during depilation, neither reference to an anti irritative potential when AlCl is combined with Sulfur. On the contrary, AlCl and Sulfur both are potential mild irritants.

Anhydrous aluminium chloride is a powerful irritant to tissue; moderately toxic by ingestion (Lewis, R. J., Sr Ed., Hawley's Condensed Chemical Dictionary. 13th ed. New York, N.Y.: John Wiley & Sons, Inc. 1997., p. 42). When used as an ubiquitous anti hyperhidrosis agent, aluminium hexahydrate may cause skin irritation in almost half of users (Knapik J J, Reynolds K, Barson J. J, Influence of an antiperspirant on foot blister incidence during cross-country hiking. Am Acad Dermatol. 1998 August; 39(2 Pt 1):202-6). AlCl contraindications are not to be used on broken or recently shaved skin (Drug Information, AlCl hexahydrate, UpToDate, Ed. B. D. Rose, Wellesley, Mass., 2004).

Sulfur may cause skin irritation as well (The Merck Index, 12^{th} Ed., Merck & Co., Inc., 1996, pp 9140), and repeated applications may cause contact dermatitis (Drug Information, Sulfur, UpToDate, Ed. B. D. Rose, Wellesley, Mass., 2004).

The present invention consists of topical compositions for the treatment and/or prevention of depilation-induced skin irritation, comprising as active ingredients:
(i) Aluminium Chloride (as herein defined, AlCl) or chemical compounds which finally release AlCl, or combinations of aluminium and chloride salts which finally release AlCl; and
(ii) Sulfur or chemical compounds which finally release Sulfur;
   and an acceptable carrier.

The subject invention also relates to methods for prevention and treatment of depilation-induced irritation by a composition comprising AlCl and Sulfur as indicated above.

The composition according to the present invention suitably comprises AlCl at a concentration from 0.0001-25.0% by weight, preferably between 0.001-10% by weight; advantageously between 0.1-5.0% by weight and Sulfur at a concentration from 0.0001-25.0% by weight, preferably between 0.001-10% by weight; advantageously between 0.1-5.0% by weight.

The compound which finally releases AlCl may be for example, but it is not restricted to:
Aluminium chloride anhydrous
Aluminium chloride hexahydrate

The carrier according to the present invention may be any conventional carrier. Said carrier may be selected from among one or more of the following materials However it should not be restricted to said materials:
Emollients—such as Isostearyl Isostearate, Isopropyl myristate.
Emulsion agents—such as Steareth-2. Steareth-20
Emulsion stabilizers—such as Cetearyl alcohol. Arachidyl behenyl alcohols&arachidylglucoside.
Occlusives—such as Dimethicone.
Humectants—such as Propylene glycol
Skin conditioning agents—such as Bisabolol.
Preservatives—such as Triclosan. Sodium benzoate

The composition according to the present invention may comprise also additional pharmaceutically and/or cosmetically acceptable compounds and/or compositions. It is to be understood that all the additional compounds and/or compositions mentioned below have to be acceptable.

The compositions according to the present invention in particular those used for the treatment and/or the prevention of depilation-induced skin irritation may further comprise, for example, one or more supplementary pharmaceutically and/or cosmetically active compounds capable of functioning in different ways to enhance the activity of AlCl and Sulfur and/or to provide other anti irritant advantages, as follows:
1. Topical antibiotics, e.g. clindamycin, tetracycline, erythromycin, etc.
2. Antifungals, e.g. triazoles, metronidazole, alyllamines, etc.
3. Non Steroidal Anti-Inflammatories, of all classes, e.g. acetic acid derivatives, oxicams, salicylates, fenemates, pyrazoles, propionic acid derivatives, bisabolol, etc.
4. Steroids of all strengths, from mild (e.g. hydrocortisone) to highly potent (clobetasol propionate).
5. Oils, e.g. silicone, dimethicone, cyclomethicone or phenyl trimethicone; siloxanes.
6. Emollients, such as urea and lactic acid.
7. Solvents, aqueous or organic, e.g. propylene glycol, polyethylene glycol, glycerol, sorbitol esters, ethanol, etc.
8. Astringents, e.g. camphor, allantoin, resorcin, etc.
9. Antioxidants, e.g. ascorbic acid and its salts, glutathione, selenium, etc.
10. Compounds that promote the natural tissue production of nitric oxide e.g. precursors such as L-arginine, or compounds that directly or indirectly cause the release of nitric oxide, e.g. glyceryl tri-nitrate.
11. Supplementary actives: e.g. alpha-Hydroxy acids, (such as glycolic acid), 5-alpha reductase inhibitors, azelaic acid, cetyl betaine, salicylic acid, resorcinol, zinc, zinc oxide, Topical Amiodarone, Topical eicosanoids, e.g. PGE₂, etc.
12. Skin care product additives, e.g. collagen, hyaluronic acid, elastin, mucopolysaccharides, etc.
13. Vitamins, e.g. Vitamin A, Vitamin C and its salts, tocopherol (vitamin E), Vitamin K and other esters of tocopherol, pyridoxine, panthenol, pantothenic acid, etc.
14. Topical Sodium-Proton inhibitors, e.g. Amiloride or its derivatives, etc;
15. Plant extracts known for their therapeutic effect, e.g. aloe vera, chamomile, candelilla wax, cucumber, forsynthia, ginseng, grape seed, guggal jojoba, lavender, lemon, manjistha, nettle root, rosemary, pumpkin seed, polygonum, sage, soy, tea tree oil, thyme, witch hazel etc.
16. Soaps, e.g. soaps containing triclosan, hexachlorophene, seed or bran oils, dermatological bars, rinsing and cleaning toners, mild skin abrasives such as aluminium oxide or polyethylene microspheres, make-ups, etc.
17. Amino acids, e.g. arginine, tryptophan, etc.; and
18. Presevatives, e.g. parabens, dehydroacetic acid, phenoxyethanol, benzoic acid, sodium benzoate, potassium sorbate.

The active agents may be formulated into various pharmaceutical and/or cosmetic compositions, e.g. a solution, a lotion, a tonic, a shampoo, a gel, a mousse, a wax, a stick, a mask, a soap, a moisturizer, a powder, a perfume, a dye, a brilliantine an aerosol, a pomade, a cream, an ointment, an emulsion or a paste.

The composition according to the present invention may be topically applied within a suitable carrier as defined above and/or within a solvent, a dissolvent, an emulgent, an extract, a liposome, solutions e.g. aqueous, alcoholic, oily, suspension; microemulsion, microcapsules, vesicles, etc.

The compositions, as used herein, means may be topically applied and suitable for administration to a human or lower animal

The composition according to the present invention may be applied also as part of a physical therapy, e.g. with ultraviolet, blue light spectrum, infrared radiation or ultrasound, etc.

The composition according to the present invention may be prepared by any conventional methods. It may be manufactured as indicated in the following example. However is should not be restricted to said example:
1. Water phase—Aluminium chloride is heated in water to 40-80° C. Mix to dissolution;
2. Sulfur is added to water at 40° C., and mixed vigorously to obtain a fine homogeneous dispersion of the Sulfur;
3. To water phase A at 40-80° C. add an emulsifier and mix the emulsion for 30 min. Cool the mixture to 40° C. and add the Sulfur dispersion, and mix to cool to room temperature (RT).

The present invention also consists in the use of the composition according to the present invention in the preparation of a remedy for the treatment of humans and animals suffering from depilation-induced irritation. Furthermore, the present invention consists in the use of the composition according to the present invention in the preparation of a remedy for the prevention of depilation-induced irritation in humans and animals.

The present invention consists also in a method for the treatment of humans and animals against depilation-induced irritation, with a composition according to the present invention. Furthermore, the present invention consists also in a method of prevention of depilation-induced irritation in humans and animals with a composition according to the present invention.

The present invention has been described in terms of preferred embodiments, but the skilled artisan will appreciate that various alterations, substitutions, omissions, and changes may be made without departing from the scope of the present invention. The amounts of said compounds being used may be varied in accordance with the specific requirements.

The present invention will now be illustrated with reference to the following Examples hereto, without being limited by them.

### EXAMPLE 1

This example describes the treatment effect of an AlCl/Sulfur composition on skin irritation. A 42 years old female subject suffering from skin irritation and redness applied on depilated areas of the legs a composition of 2.0% AlCl and 2% Sulfur within a common acceptable cosmetic base without active ingredients (aqua distillata, Arachidyl behenyl alcohol & arachidyl glucoside, benzoic acid, Cetearyl alcohol, dehydroacetic acid, dimethicone, ethylhexylglycerin, Isopropyl Myristate, Isostearyl isostearate, phenoxyethanol polyaminipropyl biguanide, propylene glycol, sodium benzoate steareth-2, steareth 20, triclosan). She was instructed to apply the cream after depilation and every 4-6 hours further as needed, twice to thrice times.

Six weeks later, after 3 depilations, the subject reported an excellent response and disappearance of symptoms upon use of the cream as prescribed.

### EXAMPLE 2

This case exemplifies the preventive topical use of the AlCl combination on face depilated areas of a 18 years old male suffering from after shave skin irritation, e.g. skin redness and scales, and the rechallenge effect after a short washout period. A composition of 2.0% AlCl and 2.0% Sulfur was applied daily after shaving within a common acceptable cosmetic base (see example 1) on the depilated area as a preventive treatment schedule.

A dramatic improvement in symptoms was reported four weeks later. The treatment was interrupted for 2 weeks, resulting in a reappearance of symptoms. Reapplying preventively the cream for one month on a daily basis resulted again in disappearance of his chronic skin irritation.

### EXAMPLE 3

This case exemplifies the short term treatment and preventive potential of topical use of the AlCl/Sulfur combination on leg depilated areas of a 17 years old female subject. The subject applied a 1.0% AlCl and 4.0% Sulfur combination in a cosmetic acceptable base (see example 1) on post depilated legs. She used to shave legs every 2-3 weeks, and suffered from chronic irritation signs. She was instructed to apply the cream after depilation and every 4-6 hours further as needed, twice to thrice times.

Two months later, disappearance of depilation-induced symptoms was reported upon regular use of the cream.

### EXAMPLE 4

This case exemplifies the long term treatment potential of topical use of the AlCl/Sulfur combination on leg depilated areas of a 29 years old female subject. The subject applied a 1.0% AlCl and 4.0% Sulfur combination in a cosmetic acceptable base (see example 1) on post depilated legs, once or twice post a monthly depilation. 3 months later she reported the disappearance of irritation signs from the cream first use, such as redness, pruritus and even pustules. The intensity of use of the cream decreased to once only after each depilation.

### EXAMPLE 5

| Anti Folliculitis Gel | CTFA/INCI Name | % w/w |
|---|---|---|
| Part A | Propylene glycol | 3.5 |
| | Hydroxyethylcellulose | 1 |
| Part B | Aluminium chloride | 0.3 |
| | hexahydrate | |
| | Sulfur | 4.0 |
| | Glycolic acid | 0.2 |
| | Magnesium ascorbyl | 0.5 |
| | phosphate | |
| | Disodium EDTA | 0.2 |
| | Sodium hyaluronate | 0.8 |
| | Sodium bisulfite | 0.2 |
| | Methyl paraben | 0.25 |
| | Cucumber extract | 0.5 |
| | Deionized water | 88.55 |

### Manufacturing Procedure

### Part A

1. Add Hydroxyethylcellulose in 50% w/w water under high stirring at 40° C.;
2. Add propylene glycol with stirring.

### Part B

1. Dissolve Sulfur in 50% w/w water with the following ingredients at 40° C.: aluminium chloride hexahydrate, sodium EDTA, glycolic acid, methyl paraben, sodium bisulfite, magnesium ascorbyl phosphate. After each ingredient mix to dissolution;
2. Add part B to part A with stirring;
3. Add sodium hyaluronate and cucumber extract with stirring.

### EXAMPLE 6

| Anti Folliculitis Lotion | CTFA/INCI Name | % w/w |
|---|---|---|
| Part A | Propylene glycol | 5.5 |
| | Xanthan gum | 0.6 |
| | Hexylene glycol | 1.5 |
| | Panthenol | 1 |
| Part B | Aluminium chloride | 1.5 |
| | hexahydrate | |
| | Polysorbate 80 | 0.4 |
| | Sulfur | 3.0 |
| | Allantoin | 0.5 |
| | 2-bromo,2-nitro-1,3diol | 0.03 |
| | Deionized water | 76.57 |
| Part C | Salycilic acid | 0.4 |
| | Alcohol 96% | 7.0 |
| | Retinyl palmitate | 1 |
| | Anthemis | 0.5 |
| | nobilis(Chamomile) | |
| | flower extract | |
| | Salva officinalis(sage) | 0.5 |
| | leaf extract | |

### Manufacturing Procedure

### Part A

1. Add xanthan gum to 50% w/w water at 40° C. under high stirring;
2. Add propylene glycol, hexylene glycol and panthenol with stirring.

### Part B

1. Dissolve in 50% w/w water the following ingredients at 40° C.: sulfur, aluminium chloride, allantoin, polysorbate 80, 2-bromo-2-nitro-1,3 diol. After each ingredient stir to dissolution.

### Part C

1. Dissolve salicylic acid and retinyl palmitate in alcohol 96% at RT;
2. Add parts B and C to part A with stirring. Cool to RT;
3. Add sage extract and chamomile extract with stirring.

### EXAMPLE 7

| Anti Folliculitis o/w | | |
|---|---|---|
| Emulsion | CTFA/INCI Name | % w/w |
| Part A | cyclomethicone | 0.5 |
| | Myristyl meristate | 3 |
| | Ethylhexyl isostearate | 2.5 |
| | Steareth 2 | 2.5 |
| | Cetearyl alcohol | 3.5 |
| | Glyceryl mono stearate | 2.5 |
| | self emilsified | |
| | Propyl paraben | 0.25 |
| Part B | Deionized water | 44 |
| | Aluminium chloride | 2.5 |
| | hexahydrate | |
| | Calcium lactate | 0.2 |
| | Methyl paraben | 0.3 |
| | Tetrasodium EDTA | 0.1 |
| | allantoin | 0.3 |
| Part C | Deionized water | 30 |
| | Propylene glycol | 5 |
| | Sulfur | 2.0 |
| | panthenol | 0.5 |
| | bisabolol | 0.35 |

### Manufacturing Procedure

1. Heat parts A and B separately to 80° C. under high stirring;
2. Prepare part C at 40° C. with stirring;
3. Add part A to part B with stirring;
4. Cool to 40° C. and add part C;
5. Cool to RT under moderate stirring.

### EXAMPLE 8

| Anti Folliculitis Soap | CTFA/INCI Name | % w/w |
|---|---|---|
| | Deionized water | 80 |
| | Aluminium chloride | 1 |
| | hexahydrate | |
| | Sulfur | 5.0 |
| | Glycerin | 4 |
| | Sodium lauryl glutamate | 2.5 |
| | PEG 150 distearate | 3.5 |
| | PEG 100 lanoline | 2.5 |
| | Sodium lactate | 0.25 |
| | Methyl paraben | 0.25 |
| | Fragrance | 0.2 |
| | Salva officinale(sage) | 0.8 |
| | leaf extract | |
| | | 100 |

### Manufacturing Procedure

1. Heat water to 80° C. under high stirring;
2. Add aluminium chloride hexahydrate to dissolution;
3. Add glycerin;
4. Add methyl parabens;
5. Add sodium lactate continue stirring;
6. Add sodium lauryl glutamate wile stirring;
7. Gradually add PEG 150 distearate;
8. Add slowly PEG 100 lanolin and continue stirring until dissolution;
9. Cool to 40° C., disperse sulfur in water and add sage extract and fragrance;
10. Cool to RT under moderate stirring.

### EXAMPLE 9

| Anti Folliculitis w/o | | |
|---|---|---|
| emulsion | CTFA/INCI Name | % w/w |
| Part A | Petrolatum | 2.5 |
| | Myristyl myristate | 3 |
| | Ozokerite | 1.5 |
| | Capric caprilic triglyceride | 4 |
| | Hydrogenated castor oil | 0.8 |
| | Glyceryl isostearate | 2.5 |
| | Polyglyceryl-3 oleate | 1 |
| | Ethylhexyl isostearate | 2.5 |
| | Sodium cetearyl sulfate | 2 |
| | Cetearyl alcohol | 5.5 |
| | Dimethicone | 1.5 |
| | Propyl parabens | 0.25 |
| Part B | Deionized water | 43.05 |
| | Aluminium chloride | 2.5 |
| | hexahydrate | |
| | Sulfur | 0.5 |
| | sodium lactate | 0.3 |
| | Methyl parabens | 0.3 |
| | Tetrasodium EDTA | 0.1 |
| | Allantoin | 0.25 |
| | Malic acid | 0.15 |
| Part C | Deionized water | 20 |
| | Propylene glycol | 5 |
| | Sulfur | 0.5 |
| | Panthenol | 0.5 |
| | Bisabolol | 0.2 |
| | Rose oil | 0.1 |

### Manufacturing Procedure

1. Heat part A and B separately to 80° C. under high stirring;
2. Prepare part C at 40° C. with stirring;
3. Add part B to part A with stirring;
4. Cool to 40° C. and add part C;
5. Cool to RT under moderate stirring.

### EXAMPLE 10

| Anti Folliculitis wash | CTFA/INCI Name | % w/w |
|---|---|---|
| | Sodium laureth sulfate | 30 |
| | 27% | |
| | lauryl glucoside 50% | 1.5 |
| | Cocamidopropyl betaine | 3.5 |
| | 35% | |
| | Aluminium chloride | 1.7 |
| | hexahydrate | |
| | Sulfur | 1.0 |
| | Calcium lactate | 0.2 |
| | Phenoxyethanol & | 0.3 |
| | Ethyl paraben & | |
| | Butyl paraben & | |
| | Metyl paraben & | |
| | Fragrance | 0.5 |
| | Deionized water | 61.3 |
| | | 100 |

### Manufacturing Procedure

1. Heat water to 80° C. and add aluminium chloride hexahydrate with stirring;
2. Add calcium lactate until dissolution;
3. Add sodium laureth sulphate to dissolution;
4. Add lauryl glycoside to dissolution;
5. Cool to 40 C., while stirring add cocamidobetaine and phenoxyethanol mixed with parabens;
6. Disperse sulfur at 40° C. and add to the mixture;
7. Cool to RT degrees and add fragrance.

### EXAMPLE 11

| Anti Folliculitis Wash | CTFA/INCI Name | % w/w |
|---|---|---|
| | Sodium laureth sulfate | 6.5 |
| | 27% | |
| | Propylene glycol | 6 |
| | Disodium | 3.5 |
| | Cocamphodiacetate | |
| | Aluminium chloride | 2 |
| | hexahydrate | |
| | Sulfur | 2.5 |
| | Methyl salicylate | 0.2 |
| | panthanol | 0.8 |
| | Aloe barbadensis(aloe | 2.5 |
| | vera) extract | |
| | 2-bromo2-nitropropane | 0.05 |
| | 1,3diol | |
| | Citric acid | 0.15 |
| | Deionized water | 63.8 |
| | Alcohol 96% | 12 |
| | | 100 |

### Manufacturing Procedure

1. At 40° C. add to water aluminium chloride hexahydrate, methyl salicylate, citric acid, propylene glycol with stirring;
2. Add sodium laureth sulfate under high stirring to dissolution;
3. Add disodium cocamphdiacetate with stirring to dissolution;
4. Disperse sulfur at 40° C. and add to the mixture;
5. Cool to RT and add alcohol 96%, panthenol, aloe vera and 2-bromo 2 nitropropane 1,3 diol with stirring.

### EXAMPLE 12

A total of 108 women at ages of 18-50 were enrolled for an open study of a cream containing 2.0% Sulfur and 2.0% AlCl as active ingredients. Subjects were recruited at 6 different sites along a geographic scale of 120 km diameter. In order to bypass changes related to environmental temperatures, the study was started within 3 days of patient recruiting. Only subjects with depilation-induced side effects were admitted. Study conclusion was at 4 weeks of use.

Subjects were instructed to use the cream at least twice after each depilation procedures (legs, arms, bikini area, etc).

The mean depilation intensity of the sample was 3.0 (37%, 54% and 9% of subjects depilated once, twice or beyond thrice, respectively). 55% of subjects employed mechanical shaving and 16% waxed their hair. At the end of the month, 65% used the cream at least twice. Data was assessed by interviewing subjects with a predefined extensive formulary of safety and efficacy of use. Assessment of efficacy was performed by documenting a self reported decrease of common side effects of folliculitis, such as redness, pruritus, folliculitis, pigmentation.

Results: Women who used the product once reported a 59% rate of satisfaction on first use. A twice at least recurrent use of the combination resulted in an overall 78% rate of satisfaction. It is concluded that the before mentioned combination achieves a high rate of success upon treatment of depilation-induced side effects. The biological effect is emphasized by the higher efficacy of the treatment on second use.

1. A topical compositions for the treatment and/or prevention of depilation-induced skin irritation, comprising as active ingredients:
(i) Aluminium Chloride (AlCl) or chemical compounds which finally release AlCl, or combinations of aluminium and chloride salts which finally release AlCl; and
(ii) Sulfur or chemical compounds which finally release Sulfur; and an acceptable carrier.

2. The topical composition according to claim 1 wherein the compounds which finally release AlCl are selected among Aluminium chloride anhydrous and Aluminium chloride hexahydrate.

3. The topical composition according to claim 1 which comprises AlCl at a concentration from 0.0001-25.0% by weight.

4. The topical composition according to claim 3 wherein the concentration of AlCl is 0.001-10.0% by weight.

5. The topical composition according to claim 4 wherein the concentration of AlCl is 0.1-5.0% by weight.

6. The topical composition according claim 1 which comprises Sulfur at a concentration from 0.0001-25.0% by weight.

7. The topical composition according to claim 6 which comprises Sulfur at a concentration from 0.001-10.0% by weight.

8. The topical composition according to claim 7 which comprises Sulfur at a concentration from 0.1-5.0% by weight.

9. The topical composition according to claim 1 wherein a carrier is any acceptable conventional carrier.

10. The topical composition according to claim 9 wherein the carrier is selected from one or more of the following materials:
Emollients,
Emulsion agents,
Emulsion stabilizers,
Occlusives,
Humectants,
Skin conditioning agents, or
Preservatives.

11. The topical composition according to claim 1 which further comprises one or more supplementary pharmaceutically and/or cosmetically active compounds capable of functioning in different ways to enhance the activity of AlCl and Sulfur and/or to provide other anti irritant advantages selected from the group consisting of:
a. Topical antibiotics, selected from the group consisting of clindamycin, tetracycline, and erythromycin,
b. Antifungals, selected from the group consisting of triazoles, metronidazole, and alyllamines,
c. Non Steroidal Antiinflammatories, selected from the group consisting of acetic acid derivatives, oxicams, salicylates, fenemates, pyrazoles, propionic acid derivatives, and bisabolol,
d. Steroids
e. Oils selected from the group consisting of silicone, dimethicone, cyclomethicone, phenyl trimethicone and siloxanes,
f. Emollients, selected from the group consisting of urea and lactic acid,
g. Solvents, selected from the group consisting of propylene glycol, polyethylene glycol, glycerol, sorbitol esters, and ethanol,
h. Astringents, selected from the group consisting of camphor, allantoin, and resorcin,
i. Antioxidants, selected from the group consisting of ascorbic acid and its salts, glutathione, and selenium,
j. Compounds that promote the natural tissue production of nitric oxide or compounds that directly or indirectly cause the release of nitric oxide,
k. Supplementary actives selected from the group consisting of alpha-Hydroxy acids, 5-alpha reductase inhibitors, azelaic acid, cetyl betaine, salicylic acid, resorcinol, zinc, zinc oxide, Topical Amiodarone, and Topical eicosanoids,
l. Skin care product additives, selected from the group consisting of collagen, hyaluronic acid, elastin, and mucopolysaccharides,
m. Vitamins selected from the group consisting of Vitamin A, Vitamin C and its salts, tocopherol, Vitamin K, esters of tocopherol, pyridoxine, panthenol, and pantothenic acid,
n. Topical Sodium-Proton inhibitors,
o. Plant extracts selected from the group consisting of aloe vera, chamomile, candelilla wax, cucumber, forsynthia, ginseng, grape seed, guggal jojoba, lavender, lemon, manjistha, nettle root, rosemary, pumpkin seed, polygonum, sage, soy, tea tree oil, thyme, and witch hazel
p. Soaps, soaps containing triclosan, hexachlorophene, seed or bran oils, dermatological bars, rinsing and cleaning toners, aluminium oxide, polyethylene microspheres, make-ups,
q. Amino acids, and
r. Presevatives, selected from the group consisting of parabens, dehydroacetic acid, phenoxyethanol, benzoic acid, sodium benzoate, potassium sorbate.

12. The topical composition according to claim 1 which is formulated into pharmaceutical and/or cosmetic compositions in the form of a solution, a lotion, a tonic, a shampoo, a perfume, a dye, a brilliantine, an aerosol, a pomade, a cream, an ointment, an emulsion or a paste.

13. The topical composition according to claim 1 which is topically applied within a suitable carrier, a solvent, a dissolvent, an emulgent, an extract, a liposome, an oily solution, an alcoholic solution, an aqueous solution, a suspension, a microemulsion, microcapsules or vesicles.

14. The topical composition according to claim 1 which may be topically applied and suitable for administration to a human or lower animal.

15. The topical composition according to claim 1 which may be part of a physical therapy and in combination with ultraviolet, blue light spectrum, infrared radiation or ultrasound.

16. A method for formulating a topical composition for the treatment of humans and animals suffering from depilation-induced irritation, the method comprising:
combining
(i) Aluminium Chloride (AlCl) or chemical compounds which finally release AlCl, or combinations of aluminium and chloride salts which finally release AlCl; and
(ii) Sulfur or chemical compounds which finally release Sulfur; and an acceptable carrier.

17. (canceled)

18. A method for the prevention and/or treatment of humans and animals suffering from depilation-induced irritation, the method comprising:
applying topically to a surface in need of such treatment a composition comprising:
(i) Aluminium Chloride (AlCl) or chemical compounds which finally release AlCl, or combinations of aluminium and chloride salts which finally release AlCl; and
(ii) Sulfur or chemical compounds which finally release Sulfur; and an acceptable carrier.

19.-22. (canceled)
